(19) United States
(12) Patent Application Publication
(10) Pub. No.: US 2014/0315225 A1
(43) Pub. Date: Oct. 23, 2014

(54) METHODS AND COMPOSITIONS FOR DETECTING THE PRESENCE OF ONE OR MORE AUTOANTIBODIES

(71) Applicants: Vanda A. Lennon, Rochester, MN (US); Thomas J. Kryzer, Mantorville, MN (US); James P. Fryer, Stewartville, MN (US); Sean J. Pittock, Rochester, MN (US); Metha Apiwattanakul, Bangkok (TH)

(72) Inventors: Vanda A. Lennon, Rochester, MN (US); Thomas J. Kryzer, Mantorville, MN (US); James P. Fryer, Stewartville, MN (US); Sean J. Pittock, Rochester, MN (US); Metha Apiwattanakul, Bangkok (TH)

(73) Assignee: MAYO FOUNDATION FOR MEDICAL EDUCATION AND RESEARCH, Rochester, MN (US)

(21) Appl. No.: 14/342,907

(22) PCT Filed: Sep. 14, 2012
(86) PCT No.: PCT/US2012/055590

§ 371 Date: Apr. 25, 2014

Related U.S. Application Data
(60) Provisional application No. 61/535,610, filed on Sep. 16, 2011.

(51) Int. Cl. G01N 33/564

(52) U.S. Cl. CPC G01N 33/564
USPC 435/7.92; 435/7.1; 435/7.9

(57) ABSTRACT

This disclosure describes methods and compositions for immunohistochemically detecting the presence of a SRP autoantibody in a biological sample.

### CROSS-REFERENCE TO RELATED APPLICATIONS

This application claims priority to U.S. Provisional Application Ser. No. 61/535,610, filed on Sep. 16, 2011. The disclosure of the prior application is considered part of (and is incorporated by reference in) the disclosure of this application.

### TECHNICAL FIELD

This disclosure generally relates to autoantibodies.

### BACKGROUND

Signal recognition particle (SRP) is a ribonucleoprotein consisting of six distinct polypeptides and one molecule of small cytoplasmic RNA. An autoantibody against an SRP polypeptide was described initially in 1986 as a marker of polymyositis. In that report, Reeves et al demonstrated that the patient's serum IgG immunoprecipitated SRP extracted from human erythroleukemia cells. Since that time, numerous case reports and series have confirmed the association of SRP autoantibodies with both polymyositis and dermatomyositis.

### SUMMARY

This disclosure describes methods and compositions for immunohistochemically detecting the presence of a SRP autoantibody in a biological sample.

In one aspect, a method of detecting the presence of a SRP autoantibody in a biological sample is provided. Such a method typically includes the steps of contacting a tissue section with a biological sample and a detectably-labeled secondary antibody under conditions in which a complex is formed between SRP polypeptides in the tissue section, and a corresponding SRP autoantibody in the biological sample, if present, and the detectably-labeled secondary antibody; and identifying a pattern of complex formation in the tissue sample by detecting the detectably-labeled secondary antibody. Generally, the presence of a pattern of complex formation is indicative of the presence of a SRP autoantibody in the biological sample, and the absence of a pattern of complex formation is indicative of the absence of a SRP autoantibody in the biological sample.

In some embodiments, the tissue section is a tissue section from gut. As described herein, the pattern of complex formation in the gut is in the cytoplasm of proximal epithelial cells in mucosa and enteric neurons in the gut wall. In some embodiments, the tissue section is a tissue section from kidney. As described herein, the pattern of complex formation in the kidney is in renal tubules. In some embodiments, the tissue section is a tissue section from brain. As described herein, the pattern of complex formation in the brain is in cytoplasm of cerebellar Purkinje, Golgi neurons and granular neurons.

Representative SRP polypeptides include, without limitation, SRP54 or SRP72. Representative biological samples include blood, serum, plasma, or spinal fluid. In some embodiments, the tissue section is from mammal tissue (e.g., mouse tissue). Typically, the presence of a SRP autoantibody in the biological sample is indicative of the presence of an autoimmune myopathy (e.g., inflammatory or non-inflammatory necrotizing myopathy), a neuropathy, a connective tissue disease, or cancer.

In another aspect, a composition is provided that includes a tissue section that exhibits a staining pattern following immunohistochemistry with a biological sample that comprises SRP autoantibodies. Particularly, when the tissue section is gut, the staining pattern is in the cytoplasm of proximal epithelial cells in mucosa and enteric neurons in the gut wall; when the tissue section is kidney, the staining pattern is in renal tubules; and/or when the tissue section is brain, the staining pattern is in cytoplasm of cerebellar Purkinje, Golgi neurons and granular neurons.

In still another aspect, a composition is provided that includes a tissue section and a description of a staining pattern that is indicative of the presence of a SRP autoantibody.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 shows immunohistochemical staining patterns for PCA-1 (anti-Yo) (A) or anti-SRP (B). In panel A, perikarya of Purkinje neurons (chunky pattern) and molecular layer neurons were immunoreactive. The granular layer is non-stained except for occasional Golgi neuronal perikaryon. In panel B, the anti-SRP IgG stained perikarya of Purkinje neurons and molecular layer neurons and yielded a “chicken wire” appearance in the perikarya of granular layer neurons. GL=granular layer, ML=molecular layer.

FIG. 2 shows muscle biopsies. Panel A shows healthy muscle fibers stained with hematoxylin and eosin; Panel B shows few necrotic fibers, with one invaded by macrophages. There is no evidence of widespread inflammation and no autoaggressive inflammatory exudate. Panel C shows a higher power magnification of the same field, showing a necrotic fiber engulfed by macrophages and two pale necrotic fibers in the upper right corner. The black arrow indicates a basophilic muscle fiber with plump nuclei. This represents a regenerating fiber. Panel D shows, with acid phosphatase staining, red-colored macrophages invading two necrotic fibers.

Like reference symbols in the various drawings indicate like elements.

### DETAILED DESCRIPTION

Signal recognition particle (SRP) autoantibodies are an under-appreciated marker of autoimmune myopathies. As reported herein, SRP autoantibodies are not restricted to inflammatory myopathies. The experiments described herein have revealed the presence of IgGs specific for SRP polypeptides in a spectrum of myopathic disorders and in association with neoplasia. Remarkably, the preliminary serological findings disclosed herein demonstrate a strong association between IgGs specific for SRP polypeptide subunits 54 and 72 and the diagnosis of non-inflammatory necrotizing myopathy (14 of 22 seropositive patients; 63.6%). Thus, the methods and compositions described herein can aid in early recognition of these disorders and in early cancer detection, which, with prompt immunosuppressive treatment, may significantly improve the clinical outcomes of these patients.

### Methods and Compositions

Methods and compositions are described herein that can be used for detecting, by immunohistochemistry, the presence of a SRP autoantibody in a biological sample. Immunohistochemical methods are well known in the art. Simply by way of non-limiting examples, see U.S. Pat. Nos. 5,073,504; 5,225,325; and 6,855,552. See, also, Dabbs, Diagnostic Immunohistochemistry, 2^{nd} Ed., 2006, Churchill Livingstone; and Chu & Weiss, Modern Immunohistochemistry, 2009, Cambridge University Press. In the present methods, a tissue section is typically contacted with a biological sample in the presence of a secondary antibody that is detectably-labeled.

The tissue sections used in the present methods are from mammals. For example, mouse tissue is routinely used in immunohistochemistry, but tissue from other rodents (e.g., rats) or other mammals (e.g., rabbits, primates, or humans) also can be used in the present methods. The tissue sections used in the methods described herein typically include gut tissue, kidney tissue, and/or brain tissue. An additional tissue section may include colon tissue.

Representative biological samples that can be used in the methods described herein include, without limitation, blood, serum, plasma, or spinal fluid. As is well known in the art, the secondary antibody is an antibody raised against the IgG of the animal species in which the primary antibody originated. In addition, detectable labels are well known in the art and include, without limitation, fluorescent labels (e.g., FITC) and enzymatic labels (e.g., alkaline phosphatase (AP) or horseradish peroxidase (HP)). The three components (i.e., the tissue section(s), the biological sample, and the detectably-labeled secondary antibody) are combined under conditions in which a complex is formed between SRP polypeptides in the tissue section, and a corresponding SRP autoantibody in the biological sample, if present, and the detectably-labeled secondary antibody. It would be understood by those skilled in the art that immunohistochemistry routinely includes steps that are not necessarily discussed herein in detail such as washing the tissue samples to remove unbound secondary antibodies and the parallel staining experiments with proper controls.

Using the detectable label and appropriate detection means, the pattern of complex formation within the tissue sections is identified. The pattern of complex formation within the tissue sections is directly related to the cellular location(s) of the antigen (e.g., an antigenic SRP polypeptide) bound by an autoantibody, when present, in the biological sample. As described herein, the presence of a particular pattern of complex formation in one or more types of tissue indicates the presence of a SRP autoantibody in the biological sample.

As reported herein, when tissue sections from the gut are used with a biological sample that includes one or more SRP autoantibodies, staining, which represents complex formation, is observed in the cytoplasm of proximal epithelial cells in mucosa and enteric neurons in the gut wall. Also as reported herein, when tissue sections from the brain are used with a biological sample containing one or more SRP autoantibodies, staining is observed in cytoplasm of cerebellar Purkinje, Golgi neurons and granular neurons. In addition, when tissue sections from kidney are used with a biological sample that contains one or more SRP autoantibodies, staining occurs in the renal tubules. Those skilled in the art would understand that the absence of a particular staining pattern (or the presence of non-specific staining) typically indicates the absence of any SRP autoantibodies in the biological sample, provided the proper controls have been performed.

To date, SRP polypeptides have been associated with polymyositis and dermatomyositis. As reported herein, the presence of a SRP autoantibody in biological samples from a number of individuals was associated with the presence of an autoimmune myopathy. The primary autoimmune myopathy observed was non-inflammatory necrotizing myopathy, but inflammatory necrotizing myopathy also was observed. The presence of a SRP autoantibody in a number of biological samples also was associated with a neuropathy, a connective tissue disease, or cancer. As used herein, SRP polypeptides refer to any of the six SRP polypeptides, identified by their molecular weight, SRP9 (kDa), SRP14, SRP19, SRP54, SRP68, and SRP72, as well as the small cytoplasmic 7SL-RNA.

In addition to the immunohistochemistry methods described herein, one or more further immunoassays can be performed, either before or after the immunohistochemistry methods. For example, in one embodiment, a Western blot may be performed using, for example, a panel of antigens known to be associated with autoantibodies, the results of which may warrant further evaluation using, for example, the immunohistochemistry methods described herein. In another embodiment, an immunohistochemistry method as described herein may be performed, followed by a Western blot in order to, for example, further confirm the specific antigens recognized by the autoantibodies in the biological sample.

Tissue sections used in immunohistochemistry are well known in the art and are commercially available from a number of companies (e.g., Asterand, Inc. (Detroit, Mich.); Euroimmun (Morris Plains, N.J.); or Imgenex (San Diego, Calif.)). Since the staining pattern described herein is novel, particularly with respect to SRP-associated autoimmune diseases, compositions including tissue sections (e.g., gut, brain, and/or kidney) are provided herein. Such tissue samples, following immunohistochemistry with a biological sample that contains one or more SRP autoantibodies, exhibit the staining pattern described herein. Alternatively, a composition is provided that includes tissue sections (e.g., gut, brain, and/or kidney) and a description of the staining pattern described herein such that, a person of ordinary skill, after performing the immunohistochemistry methods, could identify the presence of the staining pattern described herein and, thus, the likely presence of a SRP autoantibody.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### Patients

The method of patient ascertainment in this study distinguishes it from any previous study because the patients herein were not selected on the basis of having a rheumatologic or muscle disease, inflammatory or otherwise. They were identified incidentally in the course of blinded immunohistochemical testing of a large number of patients for neural-restricted IgG autoantibodies. The tested patient population had diverse subacute neurological problems and the differential diagnosis included paraneoplastic autoimmunity. In no case was SRP autoantibody testing requested. For patients seen at Mayo Clinic, clinical information including laboratory, radiological and oncological data was obtained from Mayo Clinic records. For patients seen at other institutions, information was obtained by telephone consultation and correspondence with the caring physician.

### Example 2

### Immunofluorescence Screening

The substrate for this integral component of the service serological evaluation was a frozen composite section of mouse cerebellum/midbrain, gut and kidney tissues (4 μm), prefixed 10 minutes in 10% formalin. Sera were diluted (1:240) in PBS containing 1% BSA, absorbed with bovine liver powder, applied to the substrate and washed after 40 minutes. Bound IgG was detected by applying FITC-conjugated goat-IgG reactive with all human IgG subclasses (Southern Biotechnology).

### Example 3

### Western Blot

A crude preparation of rough microsomes was isolated from mouse pancreas by the method of Walter and Blobel (Walter et al., 1983, Methods Enzymol., 96:84-93; Walter et al., 1983, Methods Enzymol., 96:682-691) and denatured by boiling (5 minutes) in sample buffer containing 2% SDS and 10% 2-mercaptoethanol. Molecular weight standards included broad range markers (Precision Plus Protein™ Standards Dual Color, BioRad). The proteins were separated electrophoretically in 10% polyacrylamide, then transferred electrophoretically to nitrocellulose (verified by 0.1% Ponceau S (Sigma) staining) Residual binding sites on nitrocellulose were blocked with 10% milk powder. Patients' sera were diluted (1:400) with blocking buffer, and applied to the transblotted proteins. After 1 hour at room temperature followed by washing, bound IgG was detected using HRP-conjugated goat anti-human IgG (Biosource). An enhanced chemiluminescence substrate (SuperSignal West Pico Chemiluminescence Substrate, Thermo Scientific (Product # 34080)) was used to detect HRP.

### Example 4

### ELISA

The presence of SRP54-IgG was evaluated by applying sera (diluted in PBS containing 10% normal goat serum, in doubling steps from 1:200) to Imulon II plates coated with recombinant human SRP54 protein (Diarect AG; 0.5 μg/mL). After holding for 2 hours at 37° C., the wells were washed three times with PBS containing sodium azide (0.02%) and goat serum (2%), and alkaline phosphatase-conjugated goat IgG specific for human IgG was added. Then, after 1 hour at 37° C., the wells were washed three times with PBS and phosphatase substrate (1 mg/ml in diethanolamine buffer) was added. The reaction product was measured photometrically 1 hour later (ELx800; Bio-Tek Instruments Inc; wavelength at 405 nm). Values greater than 150% of the mean optical density yielded by corresponding dilutions with healthy control human sera were considered positive. Positive sera were retested and titrated further as necessary to determine the endpoint dilution. Results were expressed as titer (i.e., reciprocal of the final positive dilution).

### Example 5

### Patient Antibody Purification

Mouse pancreatic lysate proteins separated by electrophoresis were trans-blotted to nitrocellulose. Bound antigenic protein was located by Western Blot staining of excised vertical edge strips. Horizontal intervening strips bearing antigens, and a control horizontal strip lacking the antigen of interest, were exposed to patients' sera (1:400 dilution absorbed with bovine liver powder, 2 hours). After washing the strips to remove non-specifically bound antibody (six times in 20 mM Tris-HCl (pH 7.6) containing 300 mM NaCl and 0.1% Tween-20), bound IgG was eluted in 0.1 M acetic acid, neutralized with 2 M Tris (pH 8.0), dialyzed 16 hr against PBS with 0.02% sodium azide, and concentrated by Amicon Ultra tube centrifugation (to a final volume <100 μL). This IgG was applied to the composite mouse tissue substrate to evaluate its immunofluorescence staining pattern.

### Example 6

### cDNA Cloning

RNA from human pancreas was reverse-transcribed to provide first-strand complementary DNA (cDNA). Gene specific primer (SRP72 R3; 5′—CCA TAT CTC ACT AGG CAG AC—3′ (SEQ ID NO:1)) and Superscript III RT (Invitrogen) were used. A gene specific primer pair (SRP72 F1; 5′—ATG GCG AGC GGC GGC AGC GG—3′ (SEQ ID NO:2), SRP72 R3; 5′—CCA TAT CTC ACT AGG CAG AC—3′ (SEQ ID NO:3)) was used to amplify cDNA. A 2 kb product was purified using Wizard SV Gel and PCR Clean-Up System (Promega), and ligated into pcDNA4/HisMax-TOPO vector that was used to transform TOP 10 competent cells. One clone identified by restriction mapping with HindIII followed by DNA sequencing was identical to human SRP72 with the exception of one frameshift mutation and a silent mutation at codon 7 (GGG to GGT). The frameshift mutation was corrected using the QuikChange Site-Directed Mutagenesis Kit (Stratagene). Plasmid DNA was used to transform XL-10 Gold Ultracompetent cells (Stratagene). Four clones identified by HindIII restriction mapping and subsequent DNA sequencing demonstrated that the frameshift mutation had been corrected.

### Example 7

### Recombinant Protein

HEK293 cell lines were transfected (FuGENE 6 Transfection Reagent; Roche) with plasmid DNA encoding SRP72 (obtained using Qiafilter Maxi Kit; Promega).

### Example 8

### Immunohistochemical Characteristics

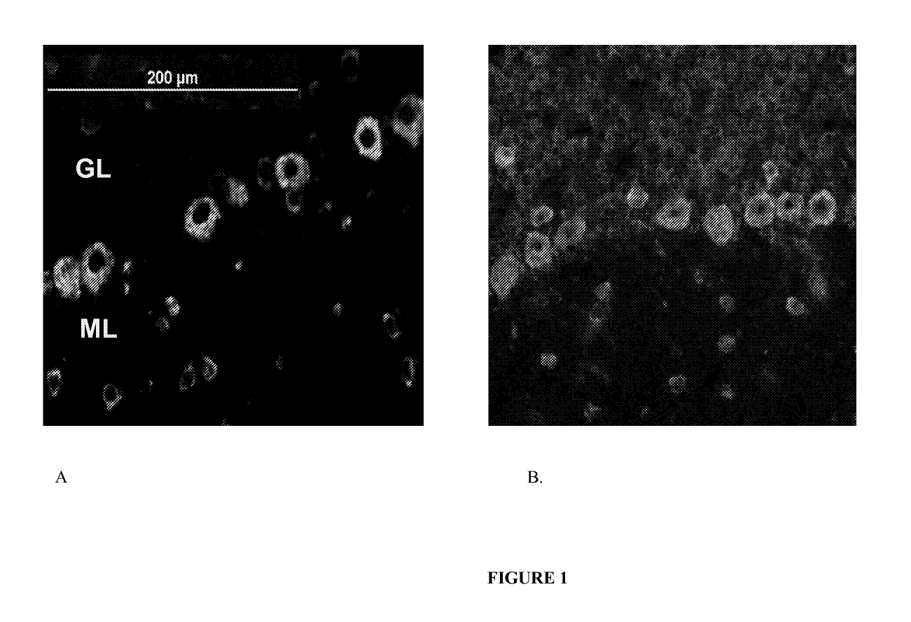

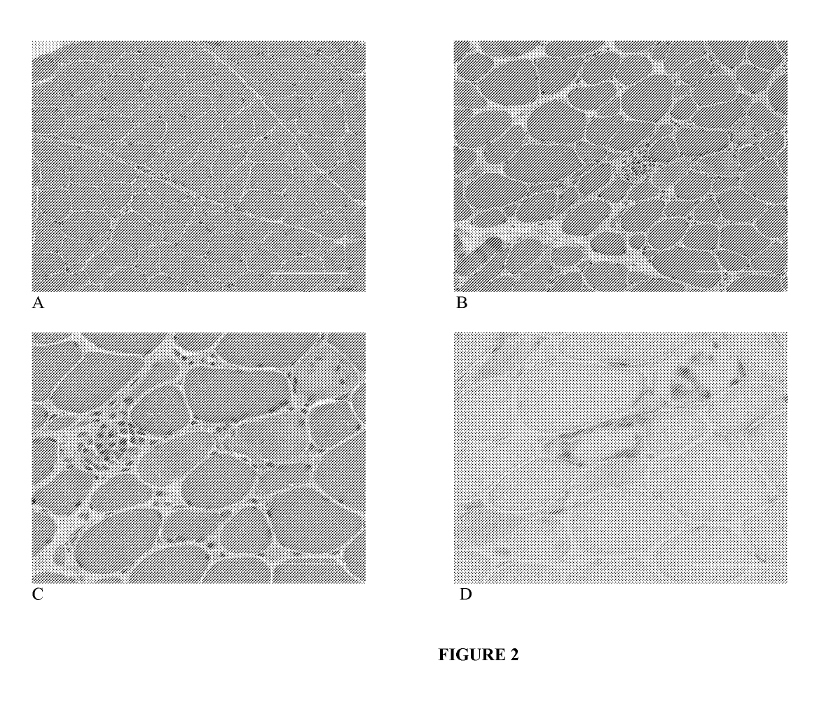

In performing the standard clinical immunofluorescence screening assay, 32 patients were identified whose serum IgG yielded striking cytoplasmic staining of cerebellar Purkinje and Golgi neurons. In this respect, the pattern resembled the PCA-1 (anti-Yo) pattern (FIG. 1) but, unlike PCA-1, this IgG also stained the cytoplasm of granular neurons (e.g., in a “chicken-wire” pattern). Furthermore, the reactivity of this IgG was not restricted to neurons; it also bound to proximal epithelial cells in the gastric mucosa and to renal tubules. The resemblance of the cytoplasmic staining in large neurons to that of PCA-1 immunoreactivity (FIG. 2) suggested that the autoantigen detected by this novel IgG might be a component of endoplasmic reticulum, as has been shown for PCA-1.

### Example 9

### Western Blot Characteristics

In further testing, IgG in these 32 patients' sera bound to microsomal antigens of molecular size consistent with known SRP subunits. Twelve bound to a 54 kD molecule, seven bound to a 72 kD molecule, and thirteen bound to both 54 and 72 kD molecules.

### Example 10

### Clinical, Serological and Neoplastic Associations, Co-Existing Autoantibodies and Demographic Data

Table 1 in Appendix A provides information on the patients evaluated herein. Twenty-eight of the 32 patients identified by immunofluorescence presented with a subacute, progressive myopathy characterized by proximal muscle weakness and, in some cases, axial muscle weakness. Bulbar musculature was involved in ten patients (36%). Four patients (14%) had Raynaud phenomenon. Among the 10 patients whose IgG bound only to SRP54, six had necrotizing myopathy, three had polymyositis, and one had non-specified myopathy. Among the five patients whose IgG bound only to SRP72, three had necrotizing myopathy and two had inflammatory myopathy. Among the thirteen patients whose IgG bound to both SRP54 and SRP72, seven had necrotizing myopathy, three had inflammatory myopathy, and three had indeterminate myopathy.

Of the remaining four identified patients, other autoimmune disorder diagnoses were neuropathy, 2; Sjögren syndrome, 1; and motor neuronopathy, 1.

### Example 11

### Histopathology

Six of the 8 Mayo Clinic patients whose muscle biopsies were reviewed had common pathological findings of necrotic and regenerating fibers with scant or no inflammatory cells (FIG. 2). The seventh patient had fiber necrosis with marked mononuclear cellular infiltrates surrounding perimysial vessels as well as endomysial connective tissue. The eighth patient had inflammatory exudates at both perimysial and endomysial sites.

### Example 12

### Oncological Findings and Other Autoimmune Disorders

Ten patients (36%) had evidence of a malignant or benign neoplasm: colon carcinoma, 1; colonic polyps, 1; endometrial carcinoma, 1; rectal tubular adenoma and thyroid adenoma, 1; Hodgkin lymphoma, 1; lung and mediastinal nodule with smoking history, 1; lung nodule, 2; and hilar plus axillary lymphadenopathy, 1; pituitary microadenoma, 1; papillary thyroid carcinoma, 1). In most patients, the diagnosis of neoplasia was made prior to myopathy onset. Thirteen patients had one or more coexisting autoantibodies: (thyroglobulin, 1; thyroperoxidase, 2; both 4); a voltage-gated potassium channel complex antibody, 3 (median 0.08 nmol/L; normal range 0.00-0.02 nmol/L); GAD65 autoantibody, 1 (0.08 nmol/L; normal range 0.00-0.02 nmol/L); ANA, 3; aquaporin-4-IgG, 1 (25 nmol/L, detected by immunoprecipitation; normal range 0.00-9.99 nmol/L).

### Example 13

### Treatment Responses

Clinical follow-up information was available for 18 patients. All but one received corticosteroid and immunosuppressant drugs. Thirteen improved after treatment, and five did not improve. Of those that did not improve, one received just corticosteroid, one received intravenous steroid and IV-Ig, one received cyclophosphamide and steroid, one received only IV-Ig, and treatment (steroid and methotrexate) was delayed in one. See Table 1 in Appendix A.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

APPENDIX A
Table 1 Demographic data, clinical presentations, evidence for neoplasia and treatment responses
in 22 SRP-IgG-positive patients

| Pa- | | | Smok- | | | | | SRP-IgG | |
|---|---|---|---|---|---|---|---|---|---|
| tient | Age/ | | ing | | Neoplasm | Muscle | Treatment/ | subunit | Other |
| No. | Sex | Race* | History^{§} | Clinical Presentation | Found | biopsy | Response | specificity | AutoAbs |
| 1 | 41/F | C | + | Proximal muscle weakness | Colon | NM | Corticosteroid/ | 54 | None |
| | | | | of lower and upper | carcinoma | | poor response (Rx | | |
| | | | | extremities, Raynaud | (7 years | | delayed) | | |
| | | | | phenomenon, no bulbar | before | | | | |
| | | | | involvement, CPK >10000 | myopathy) | | | | |
| 2 | 44/F | C | + | Progressive upper and | Colon | NM | Prednisolone/no | 54 | None |
| | | | | lower extremities | polyps (5 | | follow-up | | |
| | | | | weakness, arthritis, | years | | | | |
| | | | | dysphagia, No Raynaud | before | | | | |
| | | | | phenomenon, CPK 2824 | myopathy) | | | | |
| 3 | 48/M | C | (+) | Rapidly progressive limbs | Hodgkin | NM | IVIg and | 54, 72 | None |
| | | | | and axial weakness, | lymphoma | | corticosteroid/no | | |
| | | | | dysphagia, CPK 3485 | (3 years | | follow-up | | |
| | | | | | before | | | | |
| | | | | | myopathy) | | | | |
| 4 | 50/M | C | + | Progressive bilateral lower | Mediastinal | NM | Prednisolone/ | 54 | None |
| | | | | extremities weakness, | and hilar | | significant | | |
| | | | | Raynaud's phenomenon, | nodule | | improvement | | |
| | | | | CPK 8832 | | | | | |
| 5 | 46/F | C | + | Proximal muscle weakness | None | NM | Prednisolone/ | 54, 72 | Thyroid |
| | | | | of lower then upper | | | improved | | |
| | | | | extremities weakness, No | | | | | |
| | | | | dysphagia nor dyspnea. | | | | | |
| | | | | Hypothyroidism, CPK 1052 | | | | | |
| 6 | 66/F | C | − | Proximal muscle weakness | Not found | NM | Corticosteroid/ | 54, 72 | Thyroid |
| | | | | of lower extremities, | | | improved | | |
| | | | | Raynaud's phenomenon, | | | | | |
| | | | | Interstitial lung disease, | | | | | |
| | | | | dysphagia, CPK 7200 | | | | | |
| 7 | 40/M | ? | (+) | Proximal muscle weakness | None | Inflmm | Corticosteroid/no | 54, 72 | Thyroid |
| | | | | of upper then lower | | myositis | follow-up | | |
| | | | | extremities, dysphagia, | | | | | |
| | | | | CPK 6307 | | | | | |
| 8 | 48/F | ? | ? | Severe myopathy | None | NM | Corticosteroid/ | 54, 72 | VGKC; |
| | | | | | | | poor response | | thyroid |
| 9 | 47/M | ? | + | Rapidly progressive | None | Severe | IV corticosteroid/ | 54, 72 | None |
| | | | | proximal >distal limbs in 6 | | muscle | no follow-up | | |
| | | | | months | | atrophic | | | |
| | | | | | | fiber | | | |
| 10 | 39/F | ? | ? | Progressive proximal | None | NM | Metrotrexate and | 54 | ANA |
| | | | | muscle weakness over 4-6 | | | corticosteroid/not | | |
| | | | | weeks, wheelchair within 4 | | | improved | | |
| | | | | weeks | | | | | |
| 11 | 50/F | C | ? | 6 months painless | None | NM | IVIg, IV | 54, 72 | GAD65; |
| | | | | progressive proximal | | | corticosteroid/not | | thyroid |
| | | | | muscle weakness, now | | | improved | | |
| | | | | bed-ridden. CK 18000 | | | | | |
| 12 | 50/M | C | − | Rapidly progressive | None | Extensive | No information | 54, 72 | None |
| | | | | proximal >distal muscles | | muscle | | | |
| | | | | weakness, Bulbar | | loss with | | | |
| | | | | involvement with respiratory | | fatty | | | |
| | | | | failure. Muscle Bx: | | replacement | | | |
| | | | | extensive muscle loss with | | | | | |
| | | | | fatty replacement, cardiac | | | | | |
| | | | | involvement | | | | | |
| 13 | 70/M | ? | ? | Myopathy | No | Not | No information | 54, 72 | None |
| | | | | | information | otherwise | | | |
| | | | | | | specified | | | |
| 14 | 47/M | ? | ? | Proximal upper and lower | None | Polymyositis | Corticosteroid and | 54 | Thyroid |
| | | | | extremities weakness. | | | cyclophosphamide/ | | |
| | | | | Elevated CK | | | not improved | | |
| 15 | 39/F | ? | ? | Insidious proximal upper | None | Necrotizing | No information | 54, 72 | None |
| | | | | and lower extremities | | myopathy | | | |
| | | | | weakness. Elevated CK. | | | | | |
| | | | | Persistent pain in muscle | | | | | |
| 16 | 55/F | C | (+) | Proximal muscle weakness | Endometrial | Dermatomyositis | No information | 35 kD | ANA, |
| | | | | (lower limb) with skin rash | carcinoma | | | | dsDNA, |
| | | | | Muscle bx: perivascular and | (3 years | | | | SSA, |
| | | | | perimysial infiltrate with | before | | | | SSB |
| | | | | CD20 positive B-cell and | myopathy) | | | | |
| | | | | CD3 positive T-cell with | | | | | |
| | | | | perivascular atrophy. SLE | | | | | |
| | | | | with ANA, dsDNA, SSA, | | | | | |
| | | | | SSB+ | | | | | |
| 17 | 54/F | ? | ? | 3 yrs hx of proximal muscle | None | NM | Metrotrexate plus | 72 | None |
| | | | | weakness, feeding tube | | | low dose | | |
| | | | | and wheelchair. High CK. | | | prednisolone/ | | |
| | | | | Rx with IV steroid, IVIG | | | improved | | |
| | | | | improved can walk again | | | | | |
| | | | | Experienced couldn't get up | | | | | |
| | | | | from bed. Rx with PLEX | | | | | |
| | | | | and q 3 months, MTX + low | | | | | |
| | | | | dose prednisolone --> | | | | | |
| | | | | doing well | | | | | |
| 18 | 37/F | C | − | 2007 proximal upper and | Thyroid | inflammatory | Corticosteroid | 72 | None |
| | | | | lower extremities | adenoma, | myositis | 80 mg/day, MTX | | |
| | | | | weakness, breathing | rectal | | 2.5 mg q week/ | | |
| | | | | difficulty and blurred vision. | tubular | | improved | | |
| | | | | | adenoma | | | | |
| 19 | 77/F | ? | ? | 1 yr rapidly progressive | Lung | NM | No information | 72 | Thyroid |
| | | | | dysphagia, painless and | nodule | | | | |
| | | | | severe atrophy 4 | | | | | |
| | | | | extremities --> wheelchair | | | | | |
| | | | | bound. Muscle Bx | | | | | |
| | | | | consistent with necrotizing | | | | | |
| | | | | myositis. Anti-SRP is | | | | | |
| | | | | negative. CT chest has lung | | | | | |
| | | | | nodule but PET scan was | | | | | |
| | | | | negative. Further | | | | | |
| | | | | investigation for lung | | | | | |
| | | | | nodule ?? | | | | | |
| 20 | 45/M | ? | ? | Myopathy | None | NM | Plasmapheresis, | 72 | None |
| | | | | | | | steroid and | | |
| | | | | | | | methotrexate/ | | |
| | | | | | | | improved | | |
| 21 | 67/F | ? | + | Myopathy | Lung | IM | No information | 72 | AQP4 |
| | | | | | nodule | | | | |
| 22 | 37/M | ? | − | 2 yrs progressive proximal | Hilar and | IM | IVIg/not improved | 54 | VGKC |
| | | | | upper extremities and | axillary | | | | ANA, |
| | | | | shoulder weakness, a year | lymphadenopathy | | | | Anti-Sm, |
| | | | | later developed proximal | | | | | Anti- |
| | | | | lower extremities | | | | | RNP, |
| | | | | weakness, mild dysphagia | | | | | Anti- |
| | | | | for solid foods | | | | | SSA |
| 23 | 53/M | C | − | 2 years painless, proximal | None | IM | prednisolone 60 mg/ | 54, 72 | None |
| | | | | leg weakness w/ 20 lb wt | | | day with once | | |
| | | | | loss and CK 13,000 | | | weekly metrotexate | | |
| | | | | treatment initially | | | | | |
| 24 | 70/M | C | − | 6 months progressive | Pituitary | IM | Prednisolone and | 35, 54, 72 | VGKC |
| | | | | proximal lower extremities | macroadenoma | | cellcept | | |
| | | | | muscle weakness then | | | | | |
| | | | | upper extremities and neck | | | | | |
| | | | | flexor weakness, | | | | | |
| | | | | hoarseness and dysphagia. | | | | | |
| | | | | No rash, pain, Raynaud | | | | | |
| | | | | phenomenon. | | | | | |
| | | | | CK ~6000. EMG consistent | | | | | |
| | | | | with axial and proximal | | | | | |
| | | | | myopathy. | | | | | |
| 25 | 55/F | AA | NA | Since early 2009 severe | None | NM | Respond well to | 54 | None |
| | | | | progressive proximal | | | steroid and IVIG | | |
| | | | | muscle weakness, | | | | | |
| | | | | dysphagia & resp failure. | | | | | |
| | | | | CK 17000. | | | | | |
| 26 | 80?F | ? | NA | Proximal lower extremities | NA | NA | NA | 54 | Striational, |
| | | | | weakness | | | | | VGCC |
| | | | | | | | | | (N), |
| | | | | | | | | | GAD65 |
| 27 | 83/F | ? | (+) | Progressive severe | NA | NM | NA | 54 | None |
| | | | | generalized muscle | | | | | |
| | | | | weakness | | | | | |
| 28 | 64/F | C | − | Severe proximal muscle | Papillary | IM | Prednisolone + | 54 | None |
| | | | | weakness; CK 4500. | thyroid | | IVIG | | |
| | | | | | carcinoma | | | | |
| 29 | 50/M | ? | + | Late 2008 insidiously onset | None | NM | Metotrexate + | 54 | None |
| | | | | progressive weakness, | | | prednisolone | | |
| | | | | dysphagia need PEJ tube | | | | | |
| | | | | and loss of voice | | | | | |

*Race: C = Caucasian, AA = African American, ? = no information,
^{§}Smoking History: + = smoker, (+) = ex-smoker, − = non-smoker

What is claimed is:

1. A method of detecting the presence of a SRP autoantibody in a biological sample, comprising the steps of:
contacting a tissue section with a biological sample and a detectably-labeled secondary antibody under conditions in which a complex is formed between SRP polypeptides in the tissue section, and a corresponding SRP autoantibody in the biological sample, if present, and the detectably-labeled secondary antibody; and
identifying a pattern of complex formation in the tissue sample by detecting the detectably-labeled secondary antibody, wherein the presence of a pattern of complex formation is indicative of the presence of a SRP autoantibody in the biological sample, and wherein the absence of a pattern of complex formation is indicative of the absence of a SRP autoantibody in the biological sample.

2. The method of claim 1, wherein the SRP polypeptide is SRP54 or SRP72.

3. The method of claim 1, wherein the tissue section is a tissue section from gut.

4. The method of claim 3, wherein the pattern of complex formation in the gut is in the cytoplasm of proximal epithelial cells in mucosa and enteric neurons in the gut wall.

5. The method of claim 1, wherein the tissue section is a tissue section from kidney.

6. The method of claim 5, wherein the pattern of complex formation in the kidney is in renal tubules.

7. The method of claim 1, wherein the tissue section is a tissue section from brain.

8. The method of claim 7, wherein the pattern of complex formation in the brain is in cytoplasm of cerebellar Purkinje, Golgi neurons and granular neurons.

9. The method of claim 1, wherein the tissue section is from mammal tissue.

10. The method of claim 9, wherein the mammal tissue is mouse tissue.

11. The method of claim 1, wherein the biological sample is blood, serum, plasma, or spinal fluid.

12. The method of claim 1, wherein the presence of a SRP autoantibody in the biological sample is indicative of the presence of an autoimmune myopathy (e.g., inflammatory or non-inflammatory necrotizing myopathy), a neuropathy, a connective tissue disease, or cancer.

13. A composition comprising a tissue section that exhibits a staining pattern following immunohistochemistry with a biological sample that comprises SRP autoantibodies, wherein:
when the tissue section is gut, the staining pattern is in the cytoplasm of proximal epithelial cells in mucosa and enteric neurons in the gut wall;
when the tissue section is kidney, the staining pattern is in renal tubules; and/or
when the tissue section is brain, the staining pattern is in cytoplasm of cerebellar Purkinje, Golgi neurons and granular neurons.

14. A composition comprising a tissue section and a description of a staining pattern that is indicative of the presence of a SRP autoantibody.
